(19) United States
(12) Patent Application Publication
(10) Pub. No.: US 2013/0324439 A1
(43) Pub. Date: Dec. 5, 2013

(54) Elastic Modulus-modified MicroEnvironment microArrays (eMEArrays) and Uses Thereof

(71) Applicant: The Regents of the University of California, Oakland, CA (US)

(72) Inventors: Mark A. LaBarge, Orinda, CA (US); Chun-Han Lin, Berkeley, CA (US)

(73) Assignee: THE REGENTS OF THE UNIVERSITY OF CALIFORNIA, Oakland, CA (US)

(21) Appl. No.: 13/911,012

(22) Filed: Jun. 5, 2013

Related U.S. Application Data
(60) Provisional application No. 61/655,896, filed on Jun. 5, 2012.
(60) Provisional application No. 61/705,727, filed on Sep. 26, 2012.

(51) Int. Cl. G01N 33/50

(52) U.S. Cl. CPC G01N 33/502
USPC 506/10; 506/18; 506/32

(57) ABSTRACT

A combinatorial elastic modulus-modified microenvironment microarray (eMEArray) platform and methods for cell-based functional screening of interactions with combinatorial microenvironments. The platform and methods allow for simultaneous control of the molecular composition and the elastic modulus, and combines the use of microarray and micropatterning technologies. The eMEArrays have been used to show that the microenvironment has effects on drug-cell interactions and contributes to therapeutic response.

### CROSS-REFERENCE TO RELATED APPLICATIONS

This application is a non-provisional of and claims priority to U.S. Provisional Patent Application No. 61/655,896, filed on Jun. 5, 2012, and to U.S. Provisional Patent Application No. 61/705,727, filed on Sep. 26, 2012, both of which are hereby incorporated by reference.

### STATEMENT OF GOVERNMENTAL SUPPORT

This invention was made with government support under Grant Numbers AG033176 and AG040081 awarded by the National Institute on Aging and by Laboratory Directed Research and Development and Contract No. DE-ACO2-05CH11231 awarded by the U.S. Department of Energy. The government has certain rights in the invention.

### REFERENCE TO SEQUENCE LISTING, TABLE, OR COMPUTER PROGRAM APPENDIX

Not applicable.

### BACKGROUND OF THE INVENTION

1. Field of the Invention

The present invention relates to combinatorial cellular microarrays, fabrication and materials and methods of using these cellular microarrays, such as for functional analysis of cell and combinatorial microenvironment interactions.

2. Related Art

The interactions between cells and their surrounding microenvironment have functional consequences for cellular behaviour. For instance, on the single cell level, distinct microenvironments can impose specific differentiation, migration, and proliferation of phenotypes, and on the tissue level the microenvironment may control processes as complex as morphogenesis and tumorigenesis (Bissell, M. J. & Labarge, M. A. Context, tissue plasticity, and cancer: are tumor stem cells also regulated by the microenvironment? Cancer Cell 7, 17-23 (2005)). Not only do the cell and molecular contents of microenvironments impact the cells within them, but the elasticity (Engler, A. J., Sen, S., Sweeney, H. L. & Discher, D. E. Matrix elasticity directs stem cell lineage specification. Cell 126, 677-689 (2006)) and geometry (McBeath, R., Pirone, D. M., Nelson, C. M., Bhadriraju, K. & Chen, C. S. Cell shape, cytoskeletal tension, and RhoA regulate stem cell lineage commitment. Dev Cell 6, 483-495 (2004)) of the tissue impact the cells. Defined as the sum total of cell-cell, -ECM, and -soluble factor interactions, in addition to physical characteristics, the microenvironment is highly complex. The phenotypes of cells within a tissue are partially due to their genomic content and partially due to the combinatorial interactions with the molecular and physical components of the microenvironment. A major challenge is to link specific combinations of microenvironmental components with distinctive behaviours. The present invention provides a means for linking the microenvironment with tissue and cell functions and behaviours.

### BRIEF SUMMARY OF THE INVENTION

The present invention provides for a combinatorial microenvironment microarray (MEArray) platform and methods. In some embodiments, the MEArray platform may be used for cell-based functional screening of interactions with combinatorial microenvironments.

In other embodiments, the present invention describes methods allowing for simultaneous control of the molecular composition and the elastic modulus, and combines the use of widely available microarray and micropatterning technologies. In some embodiments, MEArray screens require as few as 10,000 cells per array, which facilitate functional studies of cell and microenvironment interactions including rare cell types such as adult progenitor cells.

In one embodiment, the substrate is a planar glass or polymer surface. It is contemplated that the substrate can be any shaped or sized surface including but not limited to beads or particles, or other substrate surfaces.

Monomers can be polymerized on the substrate surface or the surface can be coated with a polymer. In some embodiments, the polymer comprising polydimethylsiloxanes (PDMS), polyacrylamides (PA), polyurethanes, polyethylene glycol, poly(N-isopropylacrylamide), gelatin, or agarose.

In another embodiment, the present invention comprises tuning the elastic modulus of the platform polymers to mimic the stiffnesses of different tissues. For example, the elastic modulus can be tuned by altering the base/cure ratio of polymers such as polydimethylsiloxane (PDMS), or the acrylamide/bis-acrylamide ratio of polyacrylamide (PA). In some embodiments, PDMS can mimic stiffer tissues in the range of 1-10 MPa (e.g., cartilage, cornea, and arterial walls), and PA can mimic softer tissues in the range of 100 Pa-100 kPa (e.g. breast, brain, liver, and prostate).

In some embodiments, the combinatorial microenvironment platform is used to study or detect functional interactions between specific cell or cell types in a specific tissue microenvironment. In further embodiments, the effect of drugs, toxins, analytes or other environmental substances on cells in a particular tissue microenvironment can be studied.

In some embodiments, a method of screening cellular response to a drug comprising the steps of: (a) providing a combinatorial elastic modulus-modified microenvironment microarray (eMEArray) as prepared in claim 10; (b) incubating said eMEArray; (c) contacting a drug with the cells and the eMEArray; (c) detecting any change in the cell.

In other embodiments, modulating or changing a proposed therapeutic regimen based on the drug-cell interaction observed in the eMEArrays. For example, since a sensitive response of cells to Lapatinib in tissues or microenvironments having a similar elastic modulus to 40 kPa was observed and a resistant response was observed in 400 kPa eMEArrays, a therapeutic regimen of using Lapatinib in bone cancers may not be suggested if that would promote a resistant response from cells. Conversely, use of Lapatinib in soft tissues and tumors would likely promote a sensitive response.

In another embodiment, the present MEArrays and methods are used to study the interactions between drugs and cells in an array of microenvironments. Interactions of well-known cancer drugs used effectively for a specific cancer type can be studied in the microenvironment of another tissue to determine the therapeutic effect or any reduction in therapeutic effect that is due to the microenvironment.

### BRIEF DESCRIPTION OF THE DRAWINGS

FIG. 1: A flow chart of the MEArray procedure used. First, the printing substrata are prepared either with PDMS or PA. Second, the master plates are prepared and annotated in a database. Third, the MEArrays are printed and encoded with serial numbers. Fourth, culture chambers are attached, surfaces are blocks and/or rinsed, then cells are allowed to attach and unbound cells are washed away. Fifth, cells can be treated with staining or bio-assay after a period of incubation based on experimental design. Finally, images of MEArray can be obtained and analyzed by suitable scanner and software.

FIG. 2: Deposition and relative abundance of printed proteins can be verified with immunostaining prior to cell attachment. A) Antibodies that recognized type IV collagen and laminin-111 were used to verify their presence in printed features of an MEArray. B) Using an average pixel intensity analysis feature in NIH ImageJ software, the relative abundance of the two proteins across a series of dilutions, starting from a 200 μg/mL protein solution, can be qualitatively assessed. C) Phase micrograph of D920 cells attached to square-shaped features of a printed PDMS-coated MEArray.

FIG. 3: An example of an MEArray analysis using changes in keratin expression in a multipotent progenitor cell line as a functions of time and microenvironment. Each bubble represents ratios of keratin 8 and keratin 14 protein levels from 10-15 cells attached to a feature in a MEArray. Expression was determined with immunofluorescent probes. A) Shows the keratin ratios in cells just after attachment, and B) shows the keratin ratios after 24 hours on an array that was plated in parallel. The maximum concentration of both proteins was 200 m/mL and diluted 2-fold. The diameter of a bubble represents the magnitude of the log₂ ratio of keratin 8 and keratin 14 mean intensity, and the orange and white color-coding indicates values >0 and <0, respectively. F-values for one-way ANOVA and P-values from T-tests, and brackets with arrows identifying the populations compared, are shown.

FIG. 4: An example of an MEArray scan acquired using a tiled acquisition mode on a laser scanning confocal microscope. HCC1569 cells we allowed to incorporate the DNA analog EdU for 4 hour prior to fixation. DAPI (blue) and EdU (red) are shown.

FIG. 5. Functional dissection of combinatorial microenvironments. (A) MEArrays are fabricated with commonly available tools and robots. (B) Statistically significant patterns (shown as −log(P) on Z-axis) of lineage commitment by multipotent human mammary progenitor cells are observed after 24 hrs exposure to combinatorial microenvironments (>2300 in number) that were composed of 1 mammary ECM component (1-8)+1 mammary protein (a-o). [6]. (C) Very low complexity MEArrays consisting of 36 combinations of ECM were used to determine the feasibility of detecting microenvironment-determined responses to the HER2-inhibitor lapatinib. Changes in DNA synthesis, determined by EdU incorporation, after 24 hours incubation of the HCC1569 breast cancer cell line with lapatinib were measured. Result is shown as log₂(drug treated/DMSO treated), color coding is used to represent activities that more resistant or sensitive compared to cells on tissue culture plastic.

FIG. 6 is a schematic showing that the microenvironment can affect therapeutic effects via not only chemical components of microenvironment, but also physical properties.

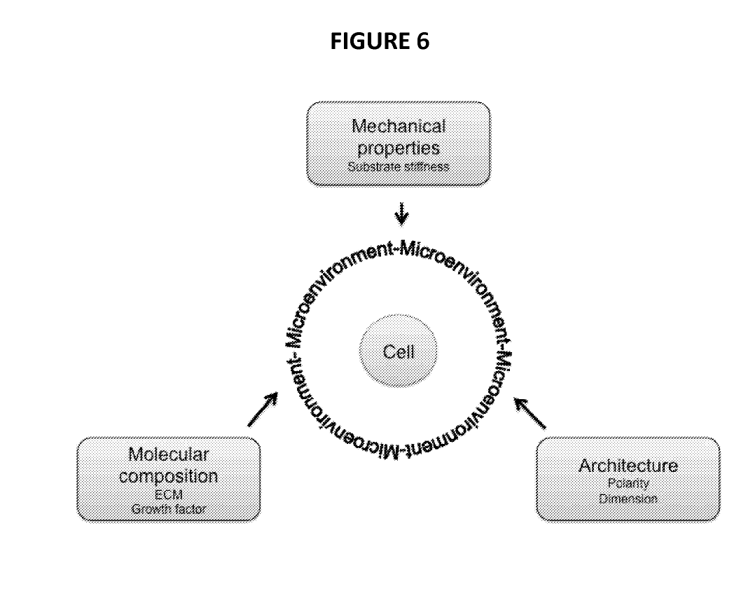

FIG. 7. HER2-targeted therapeutic response is different in breast cancer cell lines in 2D and 3D culture microenvironments as shown in Britta Weigelt, Alvin T. Lo et al. Breast Cancer Research Treat (2010). This study suggested that the HCC1569 (HER2+) cancer cell line exhibited a good dynamic range of response to Lapatinib. Thus this cell line was determined to be useful in development of the proof of principle

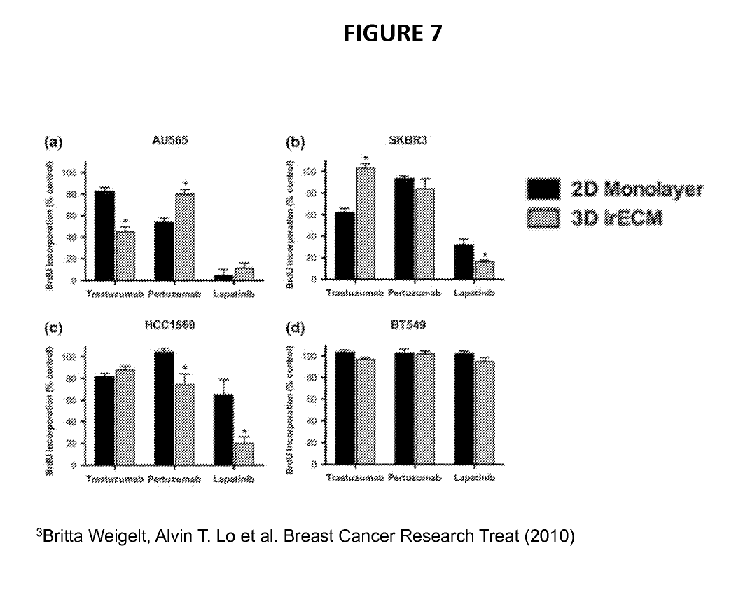

FIG. 8. A highly parallel cell-based screening platform that reveals functional effects of combinatorial microenvironments on cellular behavior. (A) Schematic showing the Microenvironment Microarrays (MEArrays), and (B) and (C) heat maps showing cellular and gene expression levels in various microenvironments on MEArrays.

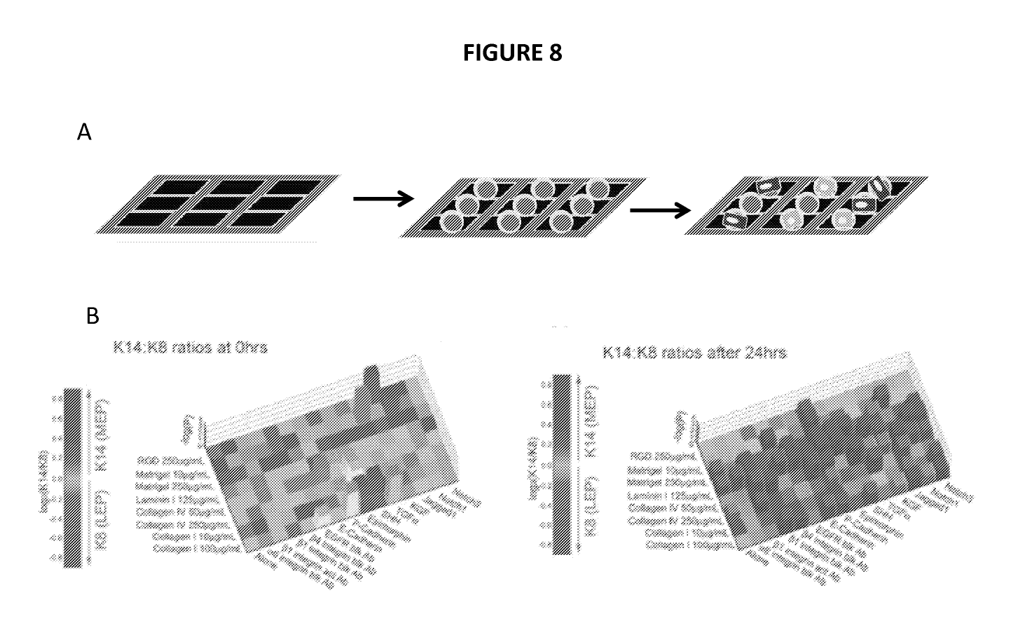

FIG. 9 shows a log scale bar of stiffness. There is a huge difference of stiffness between tissue culture dishes and physiological body tissues and Matrigel. The tissue culture dish is much stiffer than physiological microenvironments which may explain the differential growth and response of cells on plastic culture dishes as compared to Matrigel and 3D microenvironment assays. Also shown are where the presently described functionalized polyacrylamide (PA) cell culture gels having a tunable elastic modulus may fall on the scale of stiffness.

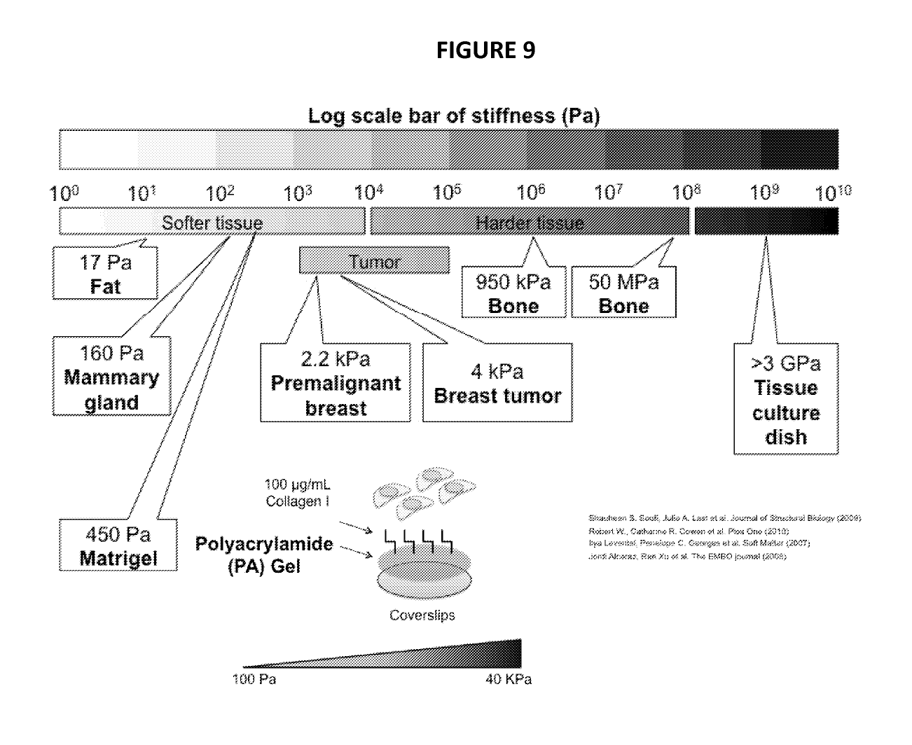

FIG. 10 is a pair of bar graphs showing that the stiffness of substrata plays a role in altering drug response of cell lines to Lapatinib. The cells were grown on plastic tissue culture dishes (2D) vs. PA gels, and 3D on top, RPMI1640 with 1% FBS and 5% Matrigel 4 days growth then 2 days with 1.5 μM Lapatinib. FIG. 10A shows verification of results in Weigelt et al BrCanRes 2009, HER2+ cell line HCC1569 is more sensitive to lapatinib in 3D Matrigel culture than on 2D tissue culture plastic, as determined by EdU encorporation. HER2− BT549 did not respond. FIG. 10B shows culturing HCC1569 on PA gels tuned to the physiological stiffness of breast, 400 Pa, yielded very similar results compared to 3D cultures. Thus the mechano-environment is an important determinant of lapatinib response.

FIG. 11 shows images of HCC1569 cells grown in 2D, PA gel, and 3D with DMSO or Lapatinib treatment. HER-2 drug response is different between 2D and 3D in HER2⁺ cells, HCC1569. BT549, a HER2⁻ cell line, was unaffected. Reproduced data as ref 3. These data show that stiffness of substrata plays a role in altering response to Lapatinib.

FIG. 12 are bar graphs showing that blocking components of the actinomyosin network impaired the modulus-dependent response to Lapatinib. The cells shown were grown for 2 days, 1 hr w/inhibitors, then 2 days with 1.5 μM lapatinib in either a 2D tissue culture dish or 3D Matrigel.

FIG. 13 is a graph showing by using intracellular flow cytometry to analyze the pHER2 and HER2 expression level of HCC1569, that cells on softer gel have higher pHER2/HER2 ratio and less EdU incorporation, vice versa, and then compared to the drug response. Thus stiffness can alter HER2 regulation and drug response. pHER2/HER2 ratios are altered in a modulus-dependent manner. (blue line) pHER2/HER2 ratios as determined with phosphor specific flow cytometry on HCC1569 cells cultured 4 days on different compliance substrata. (red line) Responses to lapatinib determined by EdU incorporation in HCC1569 as a function of substrate compliance

FIG. 14 shows bar graphs of Drug response (% EdU incorporation) of cells grown for 2 days growth, then 2 days with 1.5 μM lapatinib on (A) non-coated tissue culture dishes vs. Collagen-I coated tissue culture dishes and (B) non-coated tissue culture dishes vs. Collagen-I coated PA gels. Collagen concentration does impact lapatinib response on TC dishes, but less so on low modulus gels.

FIG. 15 is a schematic showing merging of polyacryamide gels with MEArrays. This merging allows simultaneous control of elastic modulus and molecular content.

FIG. 16 are heat maps of the resistant or sensitive drug responses of cells grown on eMEArrays where each array spot is coated with specific ECM protein combinations and grown on 400 Pa or 40 kPa PA gels.

FIG. 17 are graphs showing that the Lapatinib-response trend observed on eMEarrays corresponds with the Lapatinib-response trend validated on larger PA gels.

### DETAILED DESCRIPTION OF THE PREFERRED EMBODIMENT

### Introduction

Computational and combinatorial chemistry set the course for contemporary drug design and discovery for the last twenty years. In spite of technological advances that dramatically increased compound throughput, the rate of clinically successful therapeutics has not changed significantly. Compounds are identified largely on the behavior of tumor cell lines grown in plastic dishes, ignoring an obvious lack of accurate tissue context —for instance the stiffness of plastic (>3 GigaPa) is many orders of magnitude greater than a soft tissue like breast (˜400 Pa) or even bone (˜1 MegaPa). See Alcaraz, J., et al., Laminin and biomimetic extracellular elasticity enhance functional differentiation in mammary epithelia. EMBO J, 2008. 27(21): p. 2829-38; Levental, K. R., et al., Matrix crosslinking forces tumor progression by enhancing integrin signaling. Cell, 2009. 139(5): p. 891-906, both of which are hereby incorporated by reference. Rodent models offer an in vivo microenvironment, but large-scale in vivo screening of combinatorial chemical compound- or gene-libraries remains challenging. Hence, the inability to easily study human cells in their native microenvironment represents a significant challenge in drug discovery, and in cancer research more generally. One of the inventors with others described in Mark A. LaBarge, Celeste M. Nelson et al., “Human mammary progenitor cell fate decisions are products of interactions with combinatorial microenvironments,” Integrative Biology, 2009 January; 1(1):70-9. Epub 2008 Nov. 12, MEArrays and methods of making certain arrays with cells previously, and hereby incorporated by reference for all purposes.

Herein we describe combinatorial mimetic microenvironments fabricated in vitro for cell-based functional screening of interactions with combinatorial microenvironments of various tissues. Herein we further describe compositions and methods based upon our finding that the elastic modulus and molecular composition of the microenvironment will alter therapeutic responses. Drug responses often differ significantly between in vitro and in vivo. Identification of pathways and effectors that modulate drug resistance and sensitivity in vivo is crucial to drug design and therapeutic durability.

It has been shown that microenvironment can affect therapeutic effects via not only chemical components of microenvironment, but also physical properties. For example, myeloma cells have cell adhesion mediated drug resistance via fibronectin-β1 integrin interaction. (Jason S. Damiano, Anne E. Cress et al. “Cell adhesion mediated drug resistance (CAM-DR): role of integrins and resistance to apoptosis in human myeloma cell lines,” Blood 1999 Mar. 1; 93(5):1658-670, hereby incorporated by reference). Another example is that increasing matrix stiffness promotes chemotherapeutic resistance in hepatocellular carcinoma cell lines. (Jörg Schrader, Timothy T. Gordon-Walker et al., “Matrix stiffness modulates proliferation, chemotherapeutic response, and dormancy in hepatocellular carcinoma cells,” Hepatology 2011 April; 53 (4):1192-205. doi:10.1002/hep0.24108, hereby incorporated by reference). Recent work showed that HER2-targeted therapeutic response is different in breast cancer cell lines in 2D and 3D culture microenvironments. See Britta Weigelt, Alvin T. Lo et al. Breast Cancer Research Treat (2010).

Therefore, we sought to quantify what contributions, if any, physical and molecular properties of the microenvironment made to the effect of therapeutics on cells. We found that utilizing bioengineered culture substrata and combinatorial biology we can dissect the role played by microenvironment in drug response, and identify key points of intervention for future combination therapeutic approaches.

### DESCRIPTIONS OF THE EMBODIMENTS

In some embodiments, a combinatorial elastic modulus-modified microenvironment microarray (eMEArray, also referred to generally herein as MEArray) platform and methods for cell-based functional screening of interactions with combinatorial microenvironments. In some embodiments, the method allows for simultaneous control of the molecular composition and the elastic modulus, and combines the use of widely available microarray and micropatterning technologies. In some embodiments, eMEArray screens require as few as 10,000 cells per array, which facilitates functional studies of rare cell types such as adult progenitor cells. While entire tissue microenvironments are not completely recapitulated on the present MEArrays, however, comparison of responses in the same cell type to numerous related microenvironments, for instance pairwise combinations of extracellular matrix (ECM) proteins that characterize a given tissue, will provide insights into how microenvironmental components elicit tissue-specific functional phenotypes.

eMEArrays are amenable to time-lapsed analysis, but most often are used for end point analyses of cellular functions that are measureable with fluorescent probes. For instance, DNA synthesis, apoptosis, acquisition of differentiated states, or production of specific gene products are commonly measured.

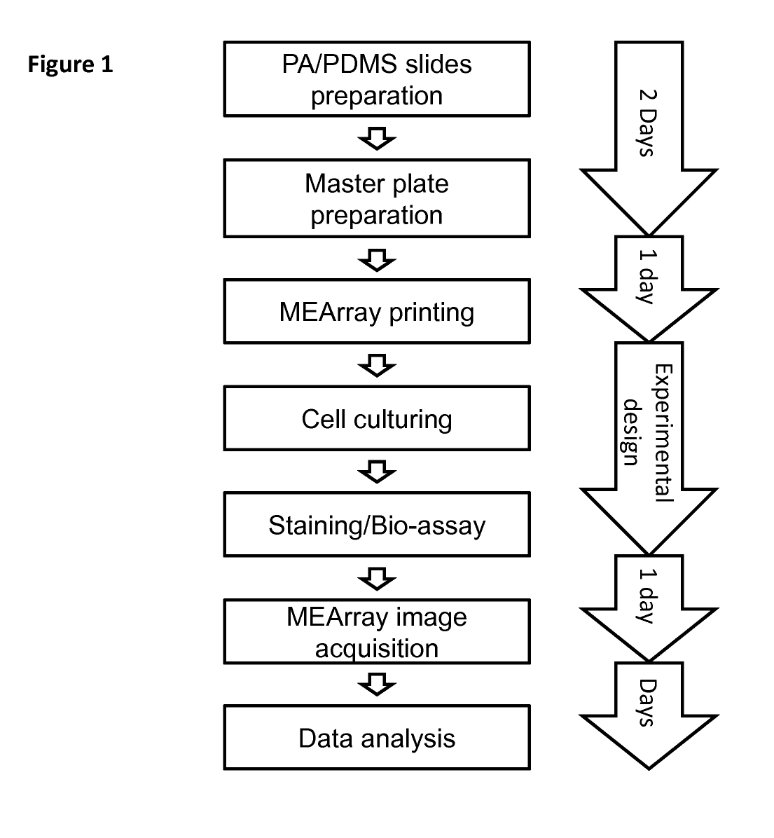

In some embodiments, the basic flow of an eMEArray experiment is to prepare substrates such as glass or plastic slides coated with the printing substrata and to prepare the master plate of proteins that are to be printed. The arrays are printed with a microarray robot, cells are allowed to attach, grow in culture, and then detected. In some embodiments, the cells are chemically fixed upon reaching the experimental endpoint. Fluorescent or colorimetric assays, imaged with traditional microscopes or microarray scanners, can be used to reveal relevant molecular and cellular phenotypes (FIG. 1).

In one embodiment, the platform comprising a substrate wherein the substrate can be a planar glass or polymer surface. It is further contemplated that the substrate can be any shaped- or sized-surface including but not limited to beads or particles, or other substrate surfaces.

Monomers can be polymerized on the substrate surface or the surface can be coated with a polymer to provide a layer of polymer on the substrate surface. In some embodiments, the polymer is polydimethylsiloxanes (PDMS), polyacrylamides (PA), polyurethanes, polyethylene glycol, poly(N-isopropylacrylamide), gelatin, or agarose.

In another embodiment, the elastic modulus of the platform polymer layer is tuned to mimic the stifihesses of different tissues. In some embodiments, the polymers may be thermocurable, UV-curable, thermoplastic or conducting polymers.

In some embodiments, the elastic modulus of the polymers can be tuned, for example, by altering the base/cure ratio of the polymers, or for example, by altering the acrylamide/bis-acrylamide ratio of PA. Various polymers and their elastic moduli are described in Kim, H. N. et al. Patterning Methods for Polymers in Cell and Tissue Engineering. Annals of Biomedical Engineering, doi:10.1007/s10439-012-0510-y (2012 June 19 online) hereby incorporated by reference for all purposes. In other embodiments, polymerization can be controlled in a gradient or variable fashion such as by the methods described in Tse and Engler, Preparation of hydrogel substrates with tunable mechanical properties. Current Protocols in Cell Biology. Chapter 10. 2010, hereby incorporated by reference.

The polymer layer of the eMEArrays can be printed using a wide variety of microenvironment components or elements such as recombinant growth factors, cytokines, and purified ECM proteins, and combinations thereof on to the polymer surface. The platform is limited only by the availability of specific reagents. Examples of some protein components include proteins including, but not limited to, Notch 1 and 3 extracellular domains, E- and P-cadherins, Jagged1, Delta-like ligand 4, Delta serrate-like peptide, sonic hedgehog, TGFβ, EGF, PDGF, FGF, IGF, IL-6, as well as integrin-blocking and -activating antibodies, collagens type I, II, III, IV, and V, laminins I and V, fibronectin, entactin, and collagenase-treated collagen 1 and 4. In some embodiments, the microenvironment components further comprising MATRIGEL.

In some embodiments, the present combinatorial microenvironment technologies are used to mimic the specialized microenvironments in which stem cells reside, called niches, which are essential to stem cell maintenance. In such embodiments, the cells used on the eMEArray are stem cells, or other kinds of progenitor cells from various tissues. In other embodiments, the present combinatorial microenvironment platform is used to study or screen cells such as tumor cells, cell lines, biopsied cells, etc.

The eMEArray method presented here enables functional analysis of cell and combinatorial microenvironment interactions. eMEArray analysis combines use of basic micropatterning technologies, cell biology, and microarray printing robots and analysis devices that are available in many multiuser facilities. eMEArray screens are compatible with most adherent cell types, though serum-free media formulations may need to be adjusted in some cases to include BSA or <1% serum, which can improve attachment. In some embodiments, this method is only limited by the availability of reagents for analyzing a given cellular function. Fluorescence-based assays are compatible with most array-based imaging systems, but colorimetric or other probe detection assays can also work well. Other variations of this method exist and support the general idea that complex microenvironments can be functionally dissected to reveal what roles individual microenvironment molecules and combinations thereof play in a variety of cell functions.

Any microdroplet printer such as a quill printer, sound oscillator printer, or microarray printer can be used to print the polymer with the cellular microenvironments. Known or suitable printers include but are not limited to microdroplet printers by Array-it (Sunnyvale, Calif.) and Shimadzu.

### EXAMPLE 1

### Preparation of eMEArrays

### 1.) Printing Substrata Preparation

The decision to use polydimethylsiloxane (PDMS)-coated or polyacrylamide (PA)-coated slides depends on the important parameters of the experimental design. The elastic modulus of both polymers can be tuned to mimic the stiffnesses of different tissues by altering the base/cure ratio of PDMS, and the acrylamide/bis-acrylamide ratio of PA. PDMS can mimic stiffer tissues in the range of 1-10 MPa (e.g. cartilage, cornea, and arterial walls), and PA can mimic softer tissues in the range of 100 Pa-100 kPa (e.g. breast, brain, liver, and prostate). See Kim, H. N. et al. Patterning Methods for Polymers in Cell and Tissue Engineering. Annals of biomedical engineering, doi:10.1007/s10439-012-0510-y (2012) hereby incorporated by reference. PDMS is inexpensive, easy to prepare, and the geometry of the printed features will be identical to the head of the printing pins. Thus the size and shape of the features can be precisely controlled using pins with different tip geometries. PDMS is more hydrophobic than PA, which causes some challenges during the cell handling and immunostaining steps, and may be incompatible with some cell lines. Because PA is a hydrogel and a native non-fouling surface, cells will only attach to spots where there are proteins that support cell adhesion. The geometry of the printed features on PA gels do not precisely follow the geometry of the pinhead; usually they become circles, due to diffusion, irrespective of the pinhead geometry that is used. Printing contact time and pin diameter parameters can be empirically determined for optimal feature size on PA gels.

Polydimethylsiloxane (PDMS)
1.1) In a disposable plastic cup combine Sylgard 184 silicone elastomer base with the curing agent at a 10:1 ratio, mix vigorously with a wooden or plastic tongue depressor then degas in a room temperature vacuum bell for 30 minutes.
1.2) Center a standard microscope slide on the vacuum actuated chuck of a spin coater, then drizzle 0.5 mL of the mixed elastomer polymer onto the center of the slide surface. Spin at 6000 RPM for 60 s.
1.3) Cure the PDMS-coated slides in a 70° C. oven or on a digital hot plate (protected from dust) for 4 hours to overnight.
1.4) Cured slides can be used immediately, or stored for several months in a slide box that is sealed within a plastic Ziploc bag and kept in a drawer. The PDMS attracts dust so it must be well protected from room air circulation.
1.5) Note: Nitrile or other non-latex, gloves must be worn when working with the PDMS elastomer kit. Incidental contact with latex gloves will inhibit PDMS polymerization.

### Polyacrylamide (PA)

1.6) NaOH etching: Place slides on heat block at 80° C. Add 1 mL 0.1N NaOH on each slide, making sure to cover the entire slide surface. Let the NaOH evaporate (a white film should form on the slide surface). Since the PA gel can only attach firmly on NaOH etched surfaces, the PA gel will detach during drying out step if the entire slide surface is not covered by NaOH. If the slide surface was not covered completely, repeat by adding 1 ml, 0.1N NaOH. Slides can then be stored at room temperature (RT) for several days. Note: an alternative to NaOH etching is to ozone- or plasma-clean the slides.
1.7) 3-Aminopropyltriethoxysilane (APES) coating: In a fume hood, place slides in a 15 mL dish, and add 300 μL APES on each NaOH etched slide. Let the APES react with the NaOH slides for 5 minutes. Exceeding this time will cause difficulty in washing out unreacted APES reagent. Wash out APES thoroughly with deionized water two to three times on both sides of the slides. If the washing is not complete, APES will be oxidized by Glutaraldehyde to form a brown deposit on slides in step 1.8.
1.8) Glutaraldehyde oxidation: Aspirate all the solutions from the slide surfaces. In each 15 cm dish, add 25 ml 0.5% glutaraldehyde in PBS. React for 30 minutes in a dark area. After 30 minutes, aspirate all the glutaraldehyde and use non-lint laboratory wipes (e.g. Kimwipes) to carefully dry the slides. Slides can then be stored at RT for up to one day.
1.9) Gel preparation: After preparing PA mixtures including acrylamide, bis-acrylamide and ddH₂O in according with the table below, degas for 30 min, and then place PA mixtures on ice to slow down polymerization. Add APS and TEMED and mix well right before making the gels. Pipet PA mixtures onto the slide surfaces and place 24 mm×50 mm, number 1 coverslips on top of the PA. Avoid pressing coverslip and glass slide together and avoid bubble formation. For gels >40,000 Pa use 100 μL, for other gels use 350 μL.

| | | | | Bis- | | | |
|---|---|---|---|---|---|---|---|
| | | Bis- | Acrylamide | Acrylamide | Deionized | | |
| Desired | Acrylamide | acrylamide | from 40% | from 2% | water | APS | TEMED |
| modulus (Pa) | % | % | stock (mL) | stock (mL) | (mL) | μL) | (μL) |
| 480 ± 160 | 3 | 0.06 | 0.75 | 0.3 | 8.95 | 100 | 10 |
| 4470 ± 1190 | 5 | 0.15 | 1.25 | 0.75 | 8 | 100 | 10 |
| 40,400 ± 2390 | 8 | 0.48 | 2 | 2.4 | 5.6 | 100 | 10 |

Adapted from^{6,7}

1.10) Let the PA gel polymerize for 2 hours, and then remove coverslips under deionized water.
1.11) Wash PA gel slides in large Coplan jars in water overnight (−8 hr) to remove unreacted acrylamides.
1.12) Dry slides in a 37° C. oven for 2-4 hours or until PA gel completely hardens.
1.13) PA gel-slides can be stored at 4° C. for one month in a sealed slide box.

### 2.) Protein Master Plate Preparation

2.1) All proteins should be aliquoted in stocks of 10× solutions in the buffers recommended by the provider and stored at −80° C. Most ECM proteins are soluble in deionized water, but the pH may need to be adjusted with drops of acetic acid. Most growth factors, cytokines, and recombinant receptor extracellular domains are prepared in PBS with BSA, but manufacturer conditions will vary. Filter the protein aliquots through a 0.45 μm 4-mm nylon syringe filter (Nalgene) prior to storage.
2.2) Design a master plate in accordance with desired protein combinations and dilutions. Adherent cells usually rely on the presence of at least one compatible ECM to mediate cell adhesion, but antibodies to cell surface epitopes can also mediate attachment sometimes. It is a good idea to add free FITC dye or a fluorphore-conjugated protein to at least one well so that arrays can be easily oriented later.
2.3) Prepare the master plate by diluting the protein combinations with printing buffer composed of 100 mM Tris-acetate/20% glycerol/0.05% Triton-100X pH5.2. Typically each well of a 384-well plate contains no more than 10 μL.
2.4) Record the contents of each well in each master plate in a tab delimited data base file and provide each master plate with a unique identification number. A six-digit date followed by the designer's initials often serves the purpose (MMDDYYinitial). Because the well volumes are small, protein aliquots can be used efficiently to generate a large numbers of replicate plates. It is recommended that master plates are stored at −80° C. and each master plate should undergo no more than two freeze-thaw cycles.

### 3.) MEArray Printing

3.1) MEArrays can be printed with most conventional microarray printing robots. Quill pin printers that use either silicone or stainless steel pins work well, but protein viscosity can be problematic. Capillary printers are ideal microarray printing robots for this application, as they work well with viscous protein solutions.
3.2) To attain good statistical power within an array, 10 to 12 replicate spots of each microenvironment is recommended. Such a design will allow comparison of activity in one microenvironment relative to another in the same array using simple T-test statistics. Dunnette's test can be used to compare activity in a control environment with other microenvironments. This design works best when a functional phenotype has been associated with the control microenvironment before performing the MEArray experiments.
3.3) Humidity should be maintained around 50%. Humidity control is important because a low humidity can dry the solution inside the pins or in the wells of the master plate causing inefficient deposition on the printing substrata. Humidity can be controlled effectively by draping the robot with non-porous plastic sheeting and using both a humidifier and a de-humidifier set to maintain 50% humidity. Cooled printing plattens can be useful for preserving some proteins, but caution must be taken to avoid condensation from forming on the slides.
3.4) Each printed array should be labeled with freezer-proof slide labels encoded with a serial number that consists of the master plate's identifier followed by a three digit number (MMDDYYinitial-nnn) As every array is used or distributed, details of their experimental treatments should be maintained in a database. Tracking the dates of printing and the numbers of freeze-thaw cycles of the master plates will help to identify the optimal conditions for maintaining reproducibility.
3.5) Printed MEArrays should be stored in sealed slide boxes at −20° C. for no more than one month. Reproducibility noticeably declines thereafter.
   4.) Culturing Mammalian Cells on eMEArrays for Functional Analysis
4.1) Attach culture chambers: To limit the volume of media and numbers of cell required to culture cells on the MEArrays, a plastic chamber is fitted to surround the printed array. For many arrays, a single chamber from a 2-chamber slide (Nunc) that contains an area of
4.2 cm² can be used. Remove the chambers from the manufactured chamber slide and cut the chambers in half with a razor blade. Use a 3 mL syringe to apply a thin bead of aquarium silicone (DAP) to the edge of a chamber and press on the surface of a MEArray. Avoid placing the applied aquarium silicone chamber on the array features.
4.2) Blocking and rinsing: MEArrays need to be well rinsed to remove unreacted monomers, which can be toxic to cells. If PDMS-coated slides were used, then the regions in between the printed features first need to be blocked with a non-fouling coating to prevent cell adhesion; incubate the arrays in 1% Pluronic F108 (BASF) in water or 2% BSA in water for 15 minutes under vacuum. PA gel slides do not require a blocking step. In all cases, rinse arrays with cell culture media three times for five minutes (media choice depends upon the cells used, but use of antibiotics is recommended regardless of media or cells). PA gels require additional 30 minutes incubation in media to rehydrate the gel.
4.3) Cell attachment: Four to five arrays can fit inside of a single 15 cm sterile Petri dish. Cover the Petri dish with a lid to keep the arrays sterile. Add half of the final media volume to the MEArray by adding the cells in media to a final concentration of 10,000 to 1,000,000 cells/mL. Cells will attach to the printed features at different rates depending on the composition of the printed microenvironment. Check for uniform attachment by viewing the arrays through an inverted stage microscope in 15 to 20 minute intervals. By gently shaking the MEArrays back and forth, cells attaching in a patterned manner can be distinguished from the floating, unattached cells.
4.4) Removal of unbound cells: On PA-coated eMEArrays, the unbound cells can be aspirated and the media can be replaced with an appropriate volume. On PDMS-coated eMEArrays, the media can never be completely removed from the well because the cells dry out and die almost immediately. Thus on PDMS-coated eMEArrays, the unbound cells must be removed by a process of successive exchanges of half of the volume of media until any unbound cells are removed, as determined by microscopic inspection. The de-wetting effect of PDMS is less prominent when serum-containing media is used compared to defined media, and when BSA is used to block the unprinted areas compared to Pluronics F108.
4.5) Cells can be cultured on eMEArrays placed inside of 15 cm Petri dishes for many days with normal media changes. Media changes on PDMS slides must be done with successive changes of half of the media volume.
4.6) Common fixatives, such as paraformaldehyde and methanol/acetone, are compatible with eMEArray systems. When staining cells on PA-coated eMEArrays, fixatives can be added and washed away just as they would be in a conventional staining procedure. However when staining cells on PDMS-coated eMEArrays, the surface must remain wet even during the fixation. Aspirate half of the media and replace with a fixative. Repeat the process a few times until the well is filled with a majority of fixative. After fixation, the fixative is gradually replaced in the same manner with blocking buffer that is appropriate for the next step of analysis.
4.7) Immunostaining is commonly used to analyze cellular functions. Staining routines will vary, but when working on the PDMS eMEArrays, one needs to perform every washing and aspiration step as above, gradually changing the solutions and never allowing the surface to de-wet. De-wetting will cause artifacts in staining
4.8) The chambers can be removed with the aid of a razor blade. Coverslips can be mounted on top of stained eMEArrays using Fluoromount-G (Southern Biotech). Detection can be performed with most multicolor fluorescence microarray scanners or on confocal microscopes with motorized tiled image acquisition modes.

### Representative Results:

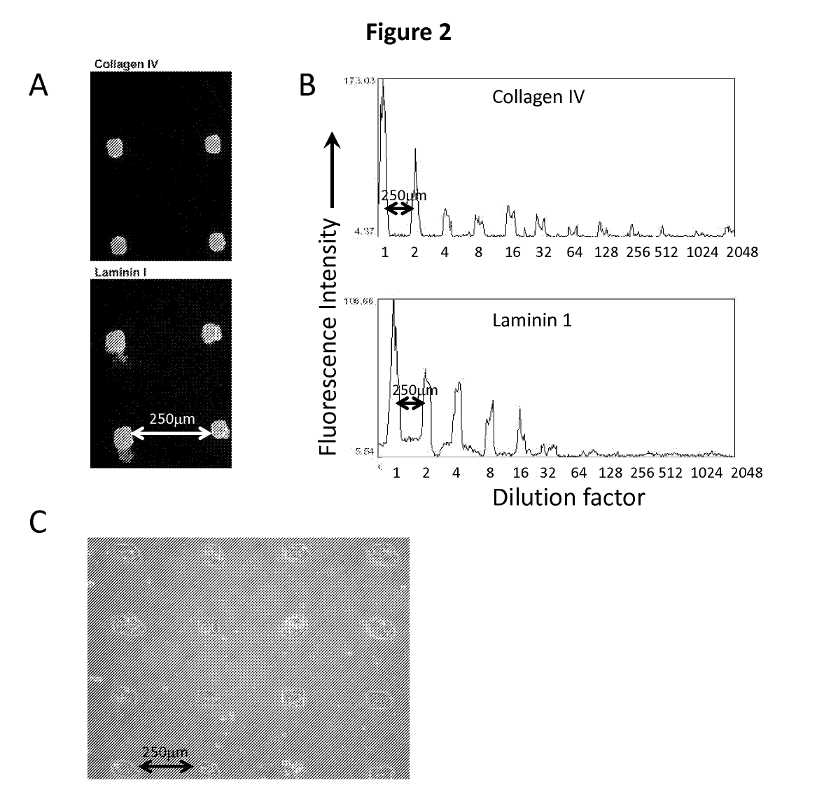

An example of patterned protein deposition on a printed PDMS-coasted eMEArray using a square-tipped silicon pins on a quill pin microarray-printing robot is shown in FIG. 2. Deposition of various proteins that are printed can be verified by immunofluorescence using antibodies (FIG. 2A). Dilutions of the protein solutions in the master plate are reflective of the amount (fluorescent intensity) that is deposited on the printing substrata surface (FIG. 2B). Cells should attach to the printed features in an obvious patterned manner (FIG. 2C).

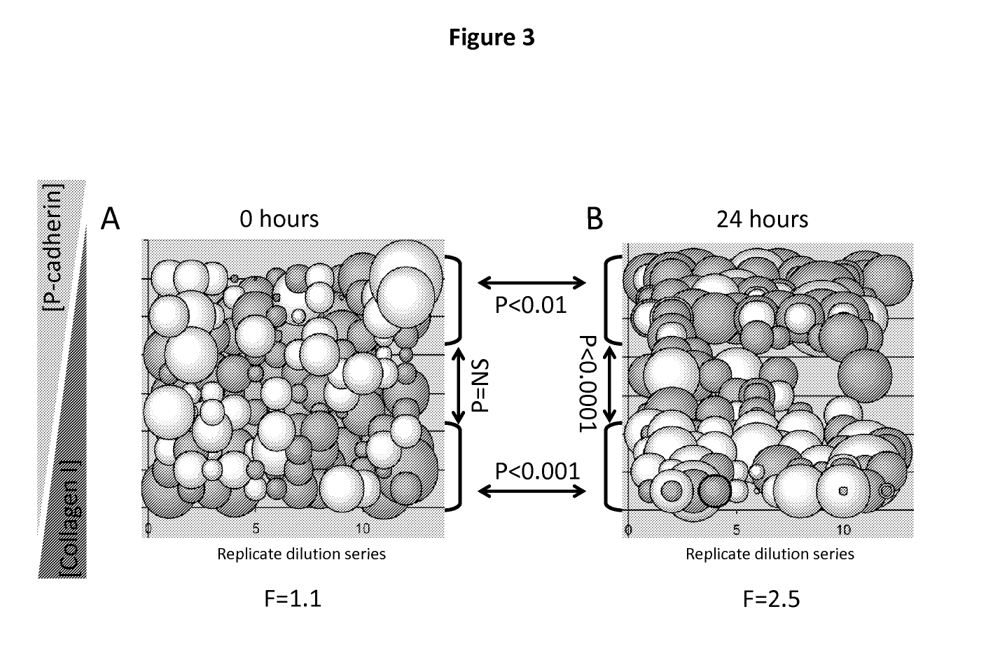

An example of an MEArray experiment showing that inverse dilutions of two microenvironment proteins elicited specific keratin expression profiles in a protein concentration-dependent manner in a human multipotent mammary epithelial progenitor cell line (D920 cells), is shown in FIG. 3. Bubble plots are useful for determining whether specific phenotypes are imposed upon cells on replicate features of a dilution series. For instance, if a particular molecule in a microenvironment causes a distinct phenotype, once the instructive component has been diluted enough into a background of a neutral ECM the phenotype should change or disappear. Immunofluorescence detection of keratin 8 and keratin 14 intermediate filament proteins was performed with an Axon 4200a (Molecular Devices) microarray scanner. Twelve replicate dilution series were printed on each MEArray, and the log₂ ratio of keratin 8 to keratin 14 mean fluorescence intensity was graphed as a bubble plot to give a realistic idea of variation and reproducibility of the signal. Shown is data from an MEArray that was fixed after cells had attached and unbound cells were washed away (FIG. 3A), and after 24 hours of culture (FIG. 3B). For this relatively small analysis, a one-way ANOVA was used to determine variance from the mean signal at each time point, and grouped two-tailed T-tests were used to determine whether the different dilutions of type I collagen and recombinant human P-cadherin caused changes in keratin expression. There was no variation from the mean among cells on the features just after attachment; however, there were significant differences in keratin expression among cells after 24 hours of exposure to the different microenvironments. T-tests verified that high type I collagen concentrations elicited higher keratin 8 expression, whereas high P-cadherin concentrations elicited a strong keratin 14 signal after 24 hours. This result was consistent with previous reports that P-cadherin-containing microenvironments will impose of K14-expressing myoepithelial phenotype on bi-potent mammary progenitor cells⁴.

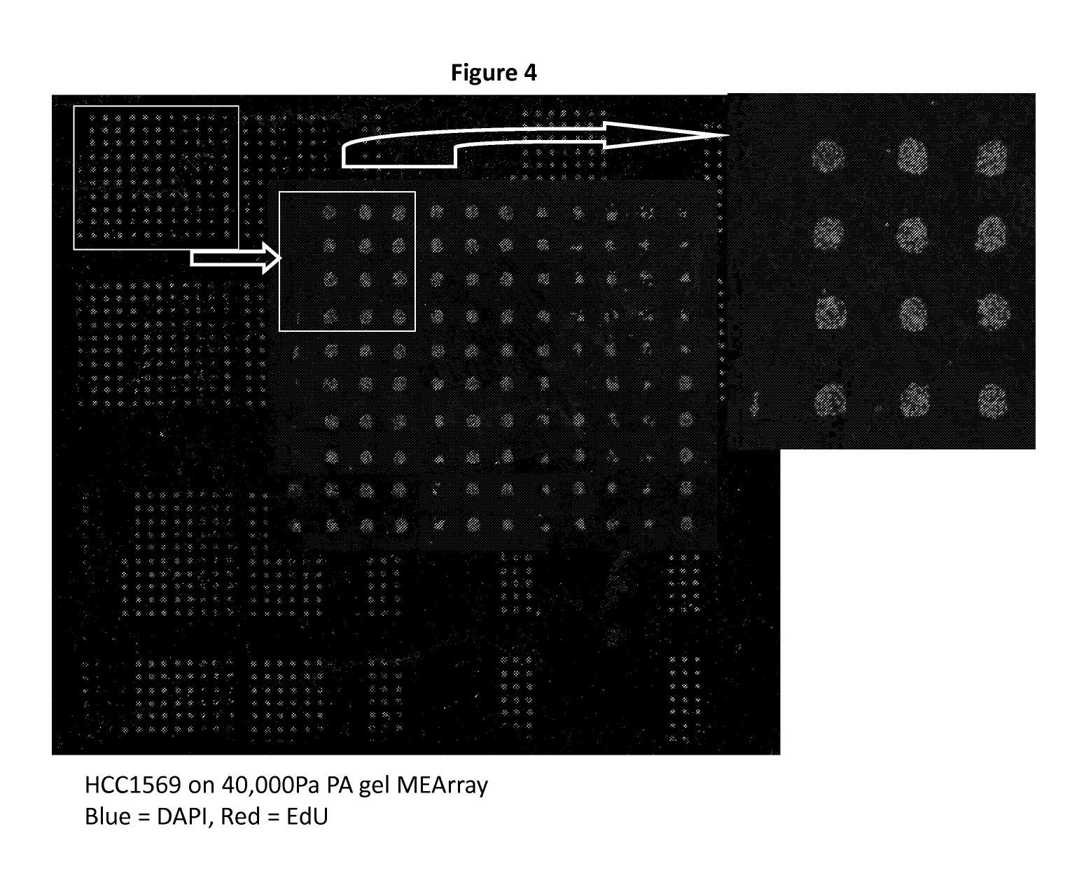

An example of an entire scanned MEArray printed on a 40,000 Pa PA gel is shown in FIG. 4.

Table 1 of specific reagents and equipment.

| Name of the reagent | Company | Catalog number | Comments (optional) |
|---|---|---|---|
| Glass slides 25 mm × | VWR | 48311-600 | |
| 75 mm | | | |
| Glass coverslips (no. 1) | VWR | 48393-241 | |
| 24 mm × 50 mm | | | |
| Staining dish (or | VWR | 25461-003 | |
| Coplan jar) | | | |
| Petri dishes (15 cm) | BD Falcon | 351058 | |
| NaOH (1.0N) | Sigma-Aldrich | S2567 | |
| APES (>98% (3- | Sigma-Aldrich | A3648 | |
| Aminopropyl)triethoxy | | | |
| silane) | | | |
| Glutaraldehyde | Sigma-Aldrich | G7651 | 50% in water |
| APS (>98% | Sigma-Aldrich | A3678 | Prepare 10% working |
| Ammonium Persulfate) | | | solution with ddH₂O |
| TEMED (N,N,N′,N′- | Sigma-Aldrich | T9281 | |
| Tetramethylethylenediamine) | | | |
| Acrylamide (40%) | Sigma-Aldrich | A4058 | |
| Bis-Acrylamide (2% | Fisher BioReagents | BP1404-250 | |
| w/v) | | | |
| 0.45 μm Syringe filter | Nalgene | 176-0045 | |
| 4-mm nylon | | | |
| FITC | Sigma-Aldrich | F4274 | |
| PDMS | Dow Corning | 3097358-1004 | Sylgard 184 |
| (polydimethylsiloxane) | | | Elastomer kit via |
| | | | Ellsworth Adhesives |
| 2-chamber slides | NUNC | 177380 | |
| Pluronic F108 | BASF | 30089186 | |
| Aquarium sealant | Dow Corning | DAP 00688 | |
| Fluormount-G | Southern Biotech | 0100-01 | |
| Disposable plastic cups | | | |
| Tongue depressors | | | |
| Nitrile gloves | | | |
| Plastic microscope slide | | | |
| boxes | | | |
| Spin coater | WS-400B- | Laurell Technologies | |
| | 6NPP/LITE | Corporation | |
| Oven | | | |
| Digital hotplate | | | |
| 384-well plates | | | A brand appropriate |
| | | | for the microarray |
| | | | robot |
| Microarray printing | | | |
| robot | | | |
| Inverted phase and | | | |
| fluorescence | | | |
| microscope | | | |
| Axon microarray | Molecular Devices | | Multiple |
| scanners | | | configurations exist |

### EXAMPLE 2

### Using MEArrays for Contextual Functional Screening of Drug-Cell Interactions

Whether developing anti-cancer drugs, improving clinical treatment regimens, or studying human cancer cells, it is important that we are able to manifest the impact of the tissue microenvironment (ME). In this Example, we describe the MEArray platform for the application of determining the functional (e.g. apoptosis, proliferation, differentiation) impact of different tissue-mimetic MEs on drug-cell interactions. We will compare tumor cell drug responses across numerous related ME conditions (differing iteratively by one component). We will develop a representation of how each ME component, and the physical properties of elasticity and shape, work together to elicit specific functional outcomes. Standard-of-care chemotherapeutics and agents that target a specific oncogenic driver (e.g., Her2) will be employed. Context-dependent changes in the antiproliferative effects (IC50 shift) on sensitive cancer cells will be determined on pair-wise combinatorial MEs that serve as mimics of different tissues.

A therapeutically relevant example of ME-modulated drug responsiveness is that HER2-expressing breast cancer cell lines were less responsive to the HER2 kinase inhibitor lapatinib in 3D Matrigel culture compared to 2D growth. This suggested that Matrigel components mediated the resistance response [See Weigelt, B., et al., HER2 signaling pathway activation and response of breast cancer cells to HER2-targeting agents is dependent strongly on the 3D microenvironment. Breast Cancer Res Treat, 2010. 122(1): p. 35-43]. The composition of Matrigel, identified by proteomic methods [Hansen, K. C., et al., An in-solution ultrasonication-assisted digestion method for improved extracellular matrix proteome coverage. Mol Cell Proteomics, 2009. 8(7): p. 1648-57], comprises ˜50 abundant ECM and growth factor proteins. By reducing the 3D ME to predetermined combinations of ME components arrayed on low stiffness substrata (Matrigel is ˜400 Pa), we can measure the responses of breast cancer cells to drugs simultaneously in different ME contexts. An MEArray can contain thousands of unique combinatorial MEs, which can be coupled with engineered and controlled surface stiffness matrices; thus, the elastic modulus (stiffness) and the molecular components used to fabricate the arrays can be chosen to mimic specific tissues. Further, culture conditions (e.g. hypoxia) can add further relevant parameters.

Mammary epithelial cultures and cancer cell lines are available from the LBNL HMEC Bank, and the Breast Cancer Cell Line Bank[Neve, R. M., et al., A collection of breast cancer cell lines for the study of functionally distinct cancer subtypes. Cancer Cell, 2006. 10(6): p. 515-27]. MEArrays are fabricated by microcontact printing with a quill-pin or a pressure-controlled capillary robot printer onto polyacrylamide (PA) or polydimethylsiloxane (PDMS)-coated glass microscope slides with combinatorial mixtures of ECM and recombinant proteins in an aqueous printing buffer using protocols developed and described above.

More recently we have switched to using PA in favor of PDMS because it is a non-fouling hydrogel with controllable stiffness. Slides are coated with PA prepared at ratios of bis/acrylamide to generate elastic moduli that mimic the stiffness of the target tissue (˜200 Pa-40,000 Pa). See Example 1, and Lin, C., J. K. Lee, and M. A. LaBarge, Fabrication and use of MicroEnvironment microArrays (MEArrays). Journal of Visual Experimentation, 2012, in press.

Initial printed arrays will consist of 2308 printed ME with a total complexity of 192 unique pairwise combinations (thus 12 replicates per ME). The total area covered by one array is approximately 2 cm² on the microscope slide surface. Examples of some protein components include but are not limited to: Notch 1 and 3 extracellular domains, E- and P-cadherins, Jagged1, Delta-like ligand 4, Delta serrate-like peptide, sonic hedgehog, TGFβ, EGF, PDGF, FGF, IGF, IL-6, as well as integrin-blocking and -activating antibodies, collagens type I, II, III, IV, and V, laminins I and V, fibronectin, entactin, collagenase-treated collagen 1 and 4 and Matrigel.

Pairwise combinations ensure that every ME is related to at least four others by one component. Nine HER2-amplified and three HER2-negative cell lines, which represent three breast cancer subtypes (four each), will be screened on Matrigel-inspired MEArrays to determine how the therapeutic responses vary as a function of microenvironment to HER2 inhibitors (Lapatinib, Trastuzumab), or chemotherapeutics (paclitaxel, doxorubicin) at published IC50 concentrations for each cell line [Konecny, G. E., et al., Activity of the dual kinase inhibitor lapatinib (GW572016) against HER-2-overexpressing and trastuzumab-treated breast cancer cells. Cancer Res, 2006. 66(3): p. 1630-9], after lh and 48 h of exposure. Cells are fixed and stained with antibodies to permit the detection of relevant markers, e.g. EdU, Caspase3, TUNEL, keratin 14/8/19, or function-specific fluorescent probes. Automated image acquisition and image analysis is conducted to quantify morphological and marker fluorescence intensity using the Zeiss 710 LSM and available software packages (e.g. ImageJ, Matlab). Ratiometric profiles will be generated using a standard microarray scanner (e.g. Axon 4200, LBNL) and the subsequent analysis will be performed using GenePix 6.0, Cluster, Treeview, and Matlab software packages.

Comparison of the mean log₂ ratio of mean fluorescence for each feature is compared to control (collagen I) to determine whether the ME constituents of that feature impose a phenotype on the cells relative to control. MEs that elicit resistance phenotypes statistically different from the control features are detected by associating a p-value to the control paired with each unique ME by Dunnette's T-test. Variance of the means is confirmed by ANOVA.

Patterns of functional phenotypes that result from the interactions of 192 different microenvironments with 12 genetically diverse cell lines and 4 different drugs at two time points will be generated. Robust evidence of that ME modulates drug responses at early stages of exposure. Genetic diversity, among cell lines, will have a stabilizing impact for identifying molecular markers.

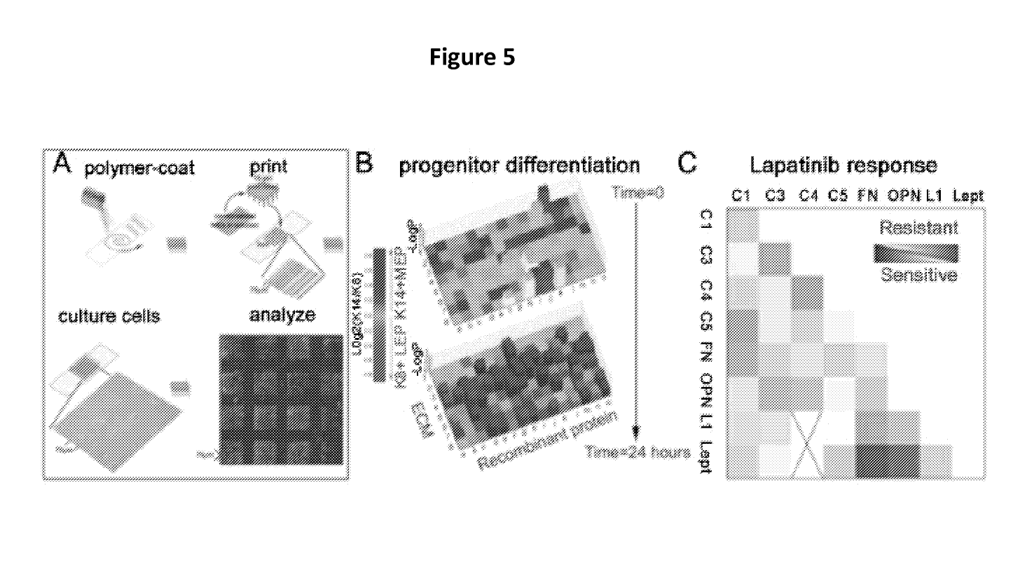

Referring now to FIG. 5, functional dissection of combinatorial microenvironments can be carried out using the eMEArrays made as described in Example 1. Very low complexity eMEArrays consisting of 36 combinations of ECM were used to determine the feasibility of detecting microenvironment-determined responses to the HER2-inhibitor lapatinib, the results described in the following Example. Changes in DNA synthesis, determined by EdU incorporation, after 24 hours incubation of the HCC1569 breast cancer cell line with lapatinib were measured (FIG. 5C). Result is shown as log₂(drug treated/DMSO treated), color coding is used to represent activities that are more resistant or sensitive compared to cells grown on tissue culture plastic.

Thus, the eMEArrays and the methods described herein may be used to identify key regulators of an ME-driven drug response phenotype which can later be validated in the 3D matrigel culture model to determine whether the response phenotypes can be predictably modulated.

### EXAMPLE 3

### The Elastic Modulus of Cell Culture Dishes and Gels and the Molecular Composition of the Microenvironment Alter Therapeutic Responses

Recent work showed that HER2-targeted therapeutic response is different in breast cancer cell lines in 2D and 3D culture microenvironments and described in Justin R. Tse, Adam J. Engler et al. Current Protocols in Cell Biology (2010), hereby incorporated by reference. Therefore, we wanted to quantify what contributions, if any, physical and molecular properties of the microenvironment made to the effect of therapeutics on cells. Utilizing bioengineered culture substrata and combinatorial biology we can dissect the role played by microenvironment in drug response, and identify key points of intervention for future combination therapeutic approaches.

Based on our previous years experience with polyacrylamide (PA) based MEArrays we fabricated MEArrays with 160 unique microenvironments meant to represent ECM and growth factor compositions at a variety of putative metastatic sites. The metastatic sites were mimicked still more by printing atop of PA gels tuned to different elastic moduli: 400 Pa, 2500 Pa, 4470 Pa, or 40,000 Pa. A detailed written and video protocol of the MEArray fabrication process is in press at the Journal of Visualized Experimentation (Lin et al., Fabrication and use of microenvironment microarrays (MEArrays), J Vis Exp. 2012 Oct. 11; (68) and hereby incorporated by reference.

During the revamping of the MEArray platform to incorporate tunable elastic modulus, we tested the impact of stiffness (elastic modulus, measured in Pascals (Pa)) alone on responses to lapatinib in HER2+ breast cancer cell line HCC 1569 and in HER2-BT549 cells. We noted that HCC1569 cultured on 2D PA tuned to the physiological stiffness of 400 Pa (Matrigel is ˜400 Pa, normal breast is 200-2400 Pa, whereas TC plastic is >3 GigaPa), crosslinked to type 1 collagen to support cell adhesion, and treated with 1.5 uM lapatinib phenocopied the response of HCC1569 grown in 3D Matrigel (FIG. 1). HER2 negative BT549 were insensitive to lapatinib in any condition.

We previously demonstrated that actinomyosin network inhibitors Y27632 (ROCK1/2 inhibitor), Blebbistatin (myosinII inhibitor), and ML-7 (myosin lightchain kinase inhibitor) altered the modulus-dependent lapatinib response on PA gels. In the case of ML-7, combination of ML-7 with Lapatinib exhibited a synergistic response that caused massive cell death on 2D PA gels. To better understand why changing the elastic modulus of the culture substrata altered sensitivity to lapatinib on PA gels we used phospho-specific intracellular flow cytometry techniques to measure the ratio of phosphorylated HER2 (pHER2, which is considered activated) to total HER2. Cells were first fixed and stained with an antibody that recognized total HER2 on the cell surface, then the cells were permeabilized and stained for pHER2 prior to multicolor analysis on a flow cytometer. HCC1569 cultured on 400 Pa, 2500 Pa, 4470 Pa, 40 KPa gels, or TC plastic while treated with 1.5 uM lapatinib for 4 days showed a higher ratio of pHER2 to total HER2 on more compliant substrata, and that ratio was inversely related to EdU incorporation (FIG. 2). This result suggested that a reason HCC 1569 are more sensitive to lapatinib on physiologically stiff substrata compared to TC plastic is that a greater proportion of HER2 molecules are in an active state, and thus are more subject to inhibitory effects of lapatinib.

Elastic Modulus of the Culture Substrata Altered HER-2-Targeted Therapeutic (Lapatinib) Response in HER-2+Breast Cancer Cell Lines.

There is a large difference of stiffness between tissue culture dishes and physiological body tissues. By tuning the stiffness of polyacrylamide (PA) gels, we are able to study drug response on different elastic modulus of substrata. Functionalized polyacrylamide (PA) cell culture gels for tunable elastic modulus were made as described above. In some embodiments, the PA gels can be tuned using the methods described in the Examples above, or using the methods known in the art including those described in Justin R. Tse, Adam J. Engler et al. Current Protocols in Cell Biology (2010).

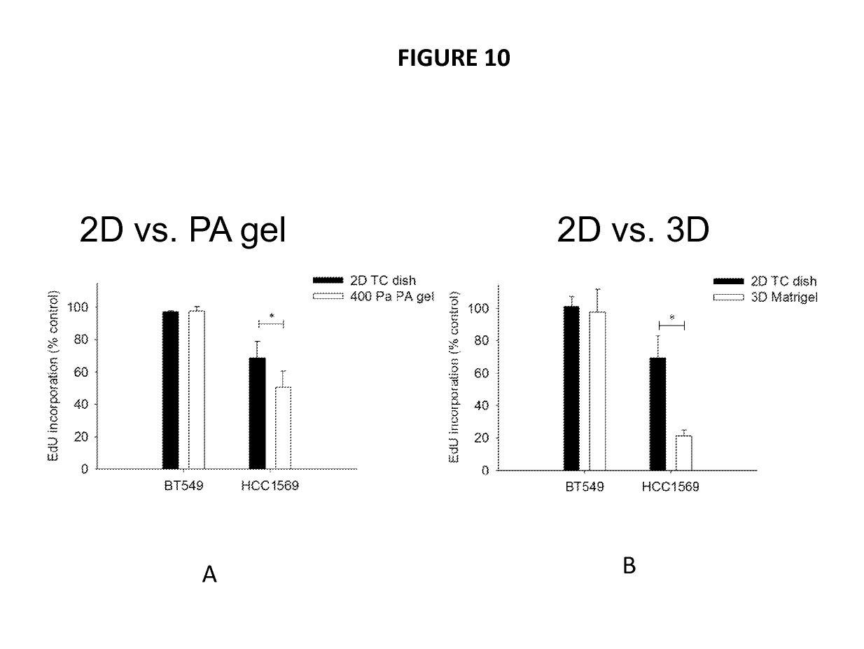

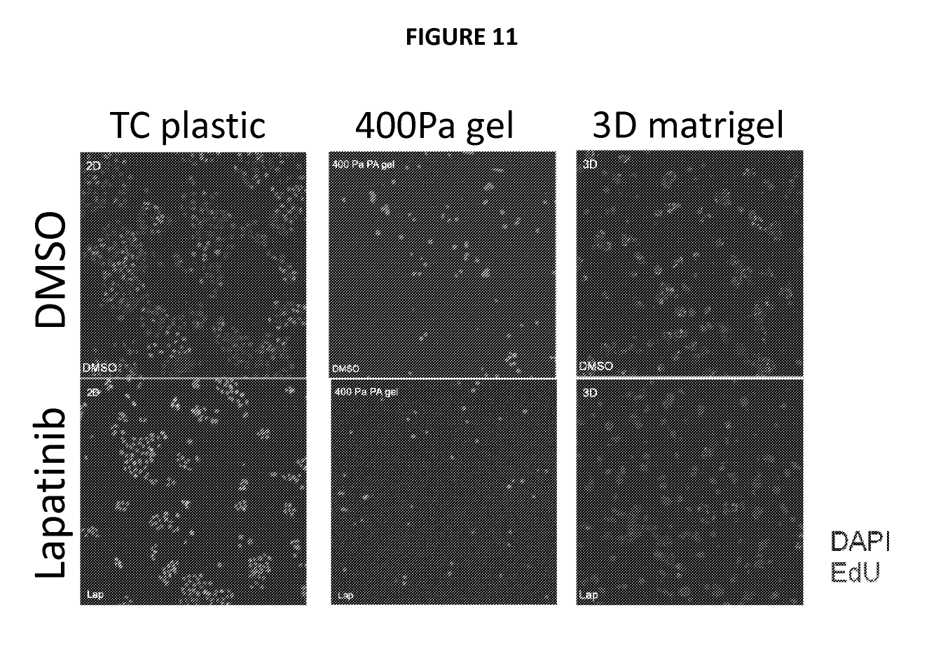

We sought to determine if HER-2 drug response was different between 2D and 3D culture environment and whether that difference is due to different substrata stiffness. Cells were grown on plastic tissue culture dishes (2D), functionalized polyacrylamide cell culture (PA) gels, and in 3D (Matrigel on top, RPMI1640 with 1% FBS and 5% Matrigel 4 days growth then 2 days with 1.5 μM Lapatinib). Referring now to FIGS. 10 and 11, HER-2 drug response is different between 2D and 3D in HER2⁺ cells, HCC1569. BT549, a HER2⁻ cell line, was unaffected. Reproduced data as ref 3. The stiffness of substrata plays a role in altering response to Lapatinib. For example, in FIG. 11, cells grown in 2D and DMSO and cells grown in the PA gels exhibited very different growth and drug response to Lapitinib as seen by the percent EdU incorporation.

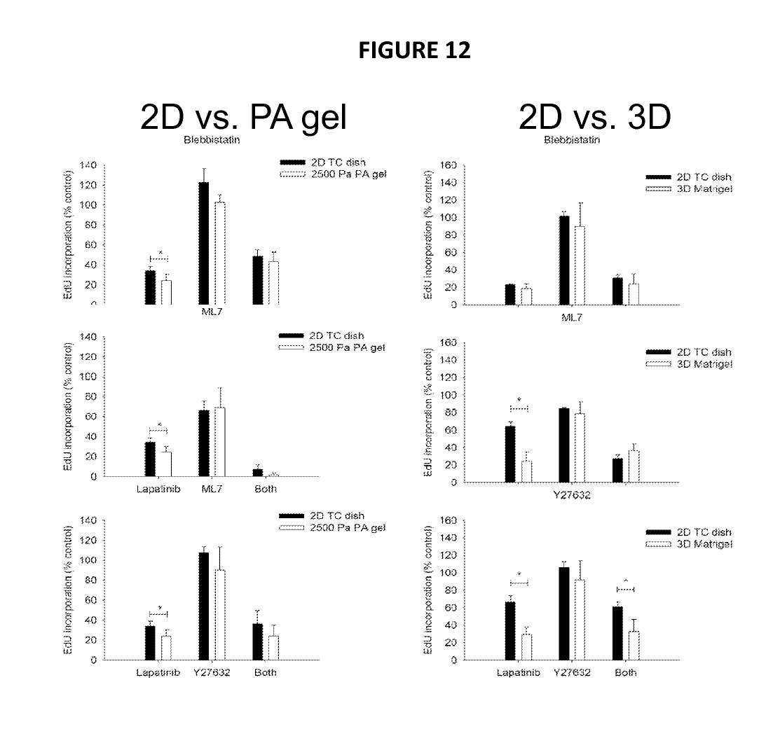

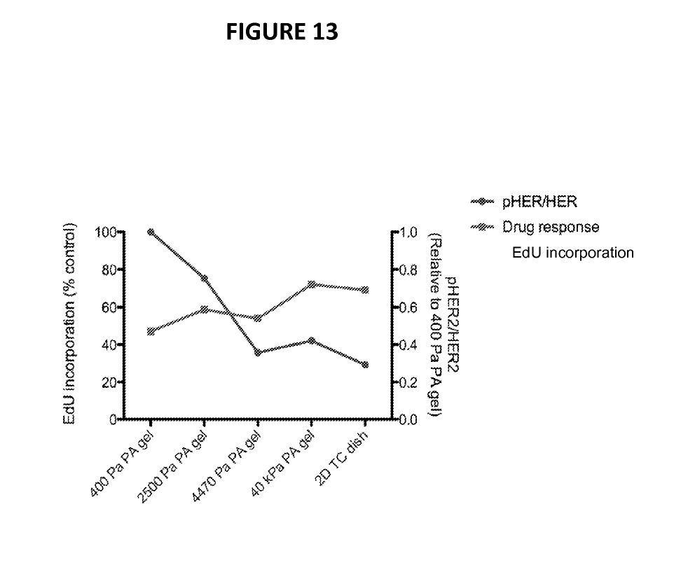

We next sought to determine if the actinomyosin network plays a role in this different drug response. Cells were grown in 2D, on PA gels, and in 3D Matrigel with 2 days growth, 1 hr w/inhibitors, then 2 days with 1.5 μM lapatinib. Referring now to FIGS. 12 and 13, blocking components of the actinomyosin network impaired the modulus-dependent response to Lapatinib Inhibition of Myosin II, Rock1/2, and MLCK were all shown to modify the modulus-dependent response on soft PA gels. However, those inhibitors did not show identical effects in 3D matrigel culture. It indicates that in the mechano-environment there is a single important factor—ECM, although other growth factors likely play roles as well.

To determine whether the actinomyosin network is involved in regulating the modulus-dependent regulation of HER2, HCC 1569 were exposed to Blebbistatin or Y27632 for 24 hours on PA gels of differing compliance. In that short time period, the ratios of pHER2/HER2 exhibited slightly different phenotypes than what was measured in the longer-term 4 day experiment. Nevertheless, modulus-dependent regulation was observed in controls, but was absent in cells treated with the actinomyosin inhibitors (FIG. 3). Thus the actinomyosin network is likely important in modulus-dependent regulation of HER2.

Abrogation of the modulus-dependent responses on compliant 2D gels by addition of actinomyosin network inhibitors suggested that the mechanosensing network could almost entirely account for results obtained on engineered 2D gel surfaces. To determine whether the 3D Matrigel context response was due only to the physiological modulus, we combined the actinomyosin network inhibitors together with lapatinib and compared the responses of cells in 3D to cells cultured on 2D TC plastic. Adding either Y27632 or ML-7 alone did not have any effect on EdU incorporation on TC plastic or in 3D, and lapatinib alone exhibited the expected context dependent responses (FIG. 4). Addition of lapatinib with Y27632 did not alter the 3D context-dependent response to lapatinib, whereas ML-7 with lapatinib eliminated any context-driven differences at the level of EdU incorporation. However, whereas we demonstrated the synergistic and toxic effect of lapatinib and ML-7 on cells grown atop 2D TC plastic or compliant PA gels in the FY2011 report, we observed no such synergistic effect in 3D (FIG. 5). Thus the modulus-dependent lapatinib response that was revealed on PA gels, is not the only microenvironmental difference that explains the differential responses in HCC1569 grown atop TC plastic versus 3D Matrigel.

Different Combinations of ECM Modified Responses to Lapatinib in HCC1569 Cells.

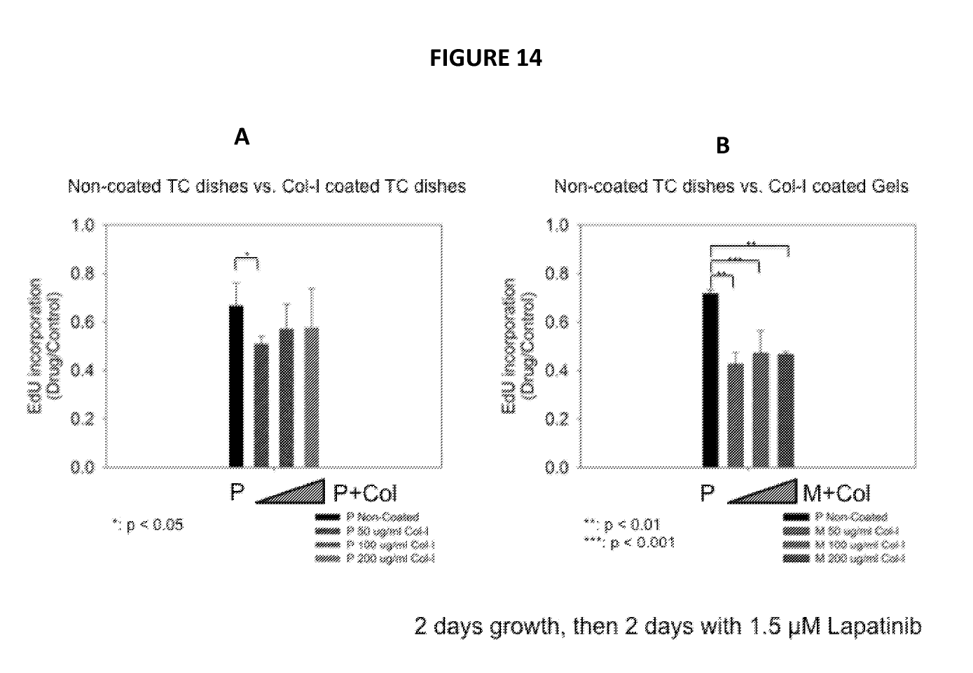

We next sought to determine if the concentration of type I collagen affected HER-2 targeted drug response. Referring now to FIG. 14, cells were subjected to non-coated tissue culture dishes, Collagen I coated tissue coated dishes for 2 days growth, then 2 days with 1.5 μM lapatinib. Collagen concentration does impact lapatinib response on TC dishes, but less so on low modulus gels.

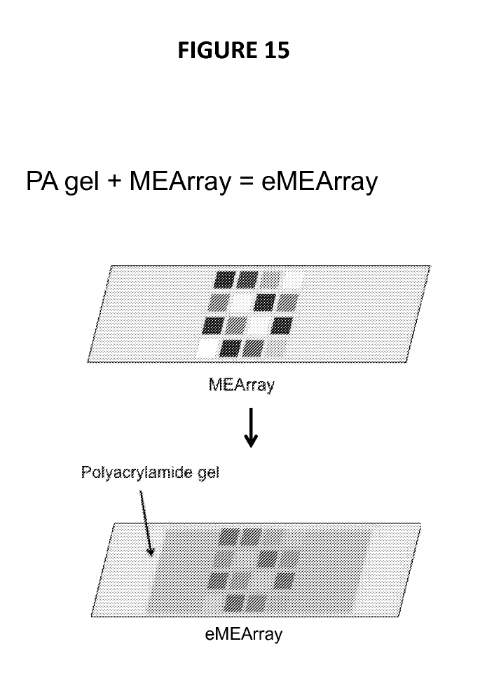

Referring now to FIG. 15, eMEarrays were made by overlaying PA gels on MEArrays as described herein. This allows for simultaneous control of elastic modulus and molecular content. The HCC1569 cells underwent 2 hrs attachment on eMEArrays, then 20 hrs growth with 1.5 μM Lapatinib for 24 hours, and EdU was added for the final 4 hours to allow for measurement of proliferation. MEArrays were fixed in methanol/acetone then were stained to allow visualization of the nuclei and the incorporated EdU. MEArrays were scanned with a microarray scanner then the total area of EdU-pixels/nucleus-pixels was determined for cells on each ME and Dunnett's tests were used to compare values from each ME to internal type 1 collagen only controls. Relative to type 1 collagen alone, the other molecular microenvironments resulted in widely varied responses to lapatinib.

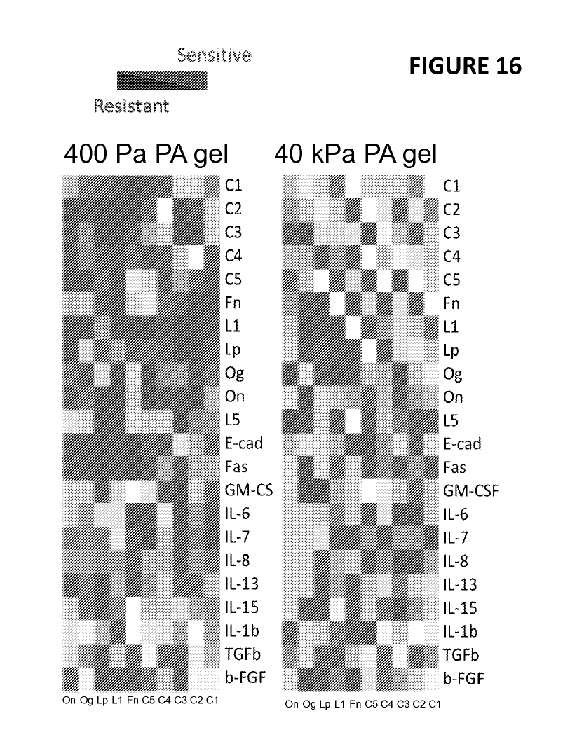

We chose for further validation five ME that exhibited strong and reproducible differences compared to type 1 collagen only on the MEArrays, they were: type II collagen, Laminin 1, type 1 Collagen+IL-8, type III collagen, and type 1 +type 4 collagens. To verify the MEArray results, larger PA gels were fabricated with those 5 different ME and HCC1569 were cultured atop of them and exposed to lapatinib (FIG. 16). Comparison of Type 1 collagen controls on 400 Pa to 40,000 Pa substrata MEArrays appears like a flat line because those ME were used to normalize the array measurements. The larger fabricated type 1 collagen control PA gels, however, recapitulated previous results (FIG. 1). Of the five experimental ME, 4/5 recapitulated MEArray results, type 3 collagen being the exception. Thus the MEArray platform demonstrated that molecular composition, in addition to substrate compliance, is an important determinant of cellular responses to lapatinib.

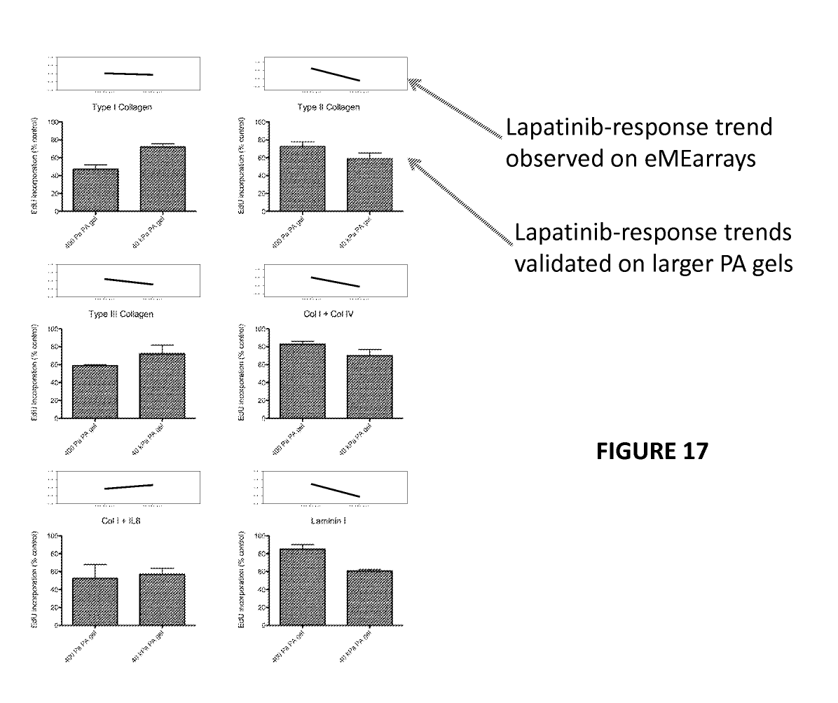

Referring now to FIGS. 16 and 17, different combinations of ME molecules affect HER-2 targeted drug responses with some cells having a resistant or a sensitive phenotype depending upon the elastic modulus and molecular content. Validation on PA gels show that different combinations of ECM modified the modulus-dependent responses to Lapatinib in HCC1569 cells. Furthermore, in FIG. 17, the Lapatinib-response trend observed on eMEarrays corresponded with the Lapatinib-response trends validated on larger PA gels.

Therefore, to summarize, HCC 1569 is inhibited more by Lapatinib in 3D culture than in 2D culture, elastic modulus of substrata plays a role in altering drug response to Lapatinib in HCC1569, and different ECM combinations imposed Lapatinib resistant or sensitive states in HCC 1569. Thus, it is contemplated that a cancer cell metastasized to a completely different organ, with a completely different microenvironmental milieu, will exhibit a different therapeutic response in the new microenvironment. Thus, these approaches using the eMEArrays and PA gels, will allow us to better understand the factors that impact drug response.

### REFERENCES

1 Bissell, M. J. & Labarge, M. A. Context, tissue plasticity, and cancer: are tumor stem cells also regulated by the microenvironment? Cancer Cell 7, 17-23 (2005).
2 Engler, A. J., Sen, S., Sweeney, H. L. & Discher, D. E. Matrix elasticity directs stem cell lineage specification. Cell 126, 677-689 (2006).
3 McBeath, R., Pirone, D. M., Nelson, C. M., Bhadriraju, K. & Chen, C. S. Cell shape, cytoskeletal tension, and RhoA regulate stem cell lineage commitment. Dev Cell 6, 483-495 (2004).
4 LaBarge, M. A. et al. Human mammary progenitor cell fate decsions are products of interactions with combinatorial microenvironments. Integrative Biology 1, 70-79 (2009).
5 Kim, H. N. et al. Patterning Methods for Polymers in Cell and Tissue Engineering. Annals of biomedical engineering, doi:10.1007/s10439-012-0510-y (2012).
6 Boudou, T., Ohayon, J., Picart, C., Pettigrew, R. I. & Tracqui, P. Nonlinear elastic properties of polyacrylamide gels: implications for quantification of cellular forces. Biorheology 46, 191-205, doi:10.3233/BIR-2009-0540 (2009).
7 Tse, J. R. & Engler, A. J. Preparation of hydrogel substrates with tunable mechanical properties. Current protocols in cell biology/editorial board, Juan S. Bonifacino . . . [et al.] Chapter 10, Unit 10 16, doi:10.1002/0471143030.cb1016s47 (2010).
8 Flaim, C. J., Chien, S. & Bhatia, S. N. An extracellular matrix microarray for probing cellular differentiation. Nat Methods 2, 119-125 (2005).
9 Soen, Y., Mori, A., Palmer, T. D. & Brown, P. O. Exploring the regulation of human neural precursor cell differentiation using arrays of signaling microenvironments. Mol Syst Biol 2, 37 (2006).
1. Minn, A. J., et al., Distinct organ-specific metastatic potential of individual breast cancer cells and primary tumors. J Clin Invest, 2005. 115(1): p. 44-55.
2. Jayanthan, A., et al., Effects of Hsp90 inhibition in neuroblastoma: analysis of drug sensitivity, target modulation and the influence of bone marrow microenvironment. J Exp Ther Oncol, 2008. 7(3): p. 183-93.
3. Matsunaga, T., et al., Interaction between leukemic-cell VLA-4 and stromal fibronectin is a decisive factor for minimal residual disease of acute myelogenous leukemia. Nat Med, 2003. 9(9): p. 1158-65.
4. Puigvert, J. C., et al., Cross-talk between integrins and oncogenes modulates chemosensitivity. Mol Pharmacol, 2009. 75(4): p. 947-55.
5. Shain, K. H., et al., Betal integrin adhesion enhances IL-6-mediated STAT3 signaling in myeloma cells: implications for microenvironment influence on tumor survival and proliferation. Cancer Res, 2009. 69(3): p. 1009-15.
6. LaBarge, M. A., et al., Human mammary progenitor cell fate decisions are products of interactions with combinatorial microenvironments. Integrative Biology, 2009. 1(1): p. 70-79.
7. Alcaraz, J., et al., Laminin and biomimetic extracellular elasticity enhance functional differentiation in mammary epithelia. EMBO J, 2008. 27(21): p. 2829-38.
8. Levental, K. R., et al., Matrix crosslinking forces tumor progression by enhancing integrin signaling. Cell, 2009. 139(5): p. 891-906.
9. Lin, C., J. K. Lee, and M. A. LaBarge, Fabrication and use of MicroEnvironment microArrays (MEArrays). Journal of Visual Experimentation, 2012.
10. Weigelt, B., et al., HER2 signaling pathway activation and response of breast cancer cells to HER2-targeting agents is dependent strongly on the 3D microenvironment. Breast Cancer Res Treat, 2010. 122(1): p. 35-43.
11. Hansen, K. C., et al., An in-solution ultrasonication-assisted digestion method for improved extracellular matrix proteome coverage. Mol Cell Proteomics, 2009. 8(7): p. 1648-57.
12. Neve, R. M., et al., A collection of breast cancer cell lines for the study of functionally distinct cancer subtypes. Cancer Cell, 2006. 10(6): p. 515-27.
13. Konecny, G. E., et al., Activity of the dual kinase inhibitor lapatinib (GW572016) against HER-2-overexpressing and trastuzumab-treated breast cancer cells. Cancer Res, 2006. 66(3): p. 1630-9.
14. Perez, O. D., et al., Activation of the PKB/AKT pathway by ICAM-2. Immunity, 2002. 16(1): p. 51-65.
15. Irish, J. M., et al., Single cell profiling of potentiated phospho-protein networks in cancer cells. Cell, 2004. 118(2): p. 217-28.
16. Caruana, R., Multitask learning. Machine Learning, 1997. 28(1): p. 41-75.
17. Yang, X., S. Kim, and E. Xing. Heterogeneous multitask learning with joint sparsity constraints. in 23rd Neural information processing systems. 2009.
18. Donoho, D., For most large underdetermined systems of linear equations the minimal l(l)-norm solution is also the sparsest solution. Communications on Pure and Applied Mathematics, 2006. 59: p. 797-829.
19. Tabshirani, R., Regression shrinkage and selection via LASSO. Journal of the Royal Statistical Society. Series B 1994. 58: p. 267-288.
20. Zhang, K., J. Gray, and B. Parvin, Sparse multitask regression for identifying common mechanism of response to therapeutic targets. Bioinformatics (ISMB), 2010. 26(12): p. 97-105.
1. Jason S. Damiano, Anne E. Cress et al. “Cell adhesion mediated drug resistance (CAM-DR): role of integrins and resistance to apoptosis in human myeloma cell lines,” Blood 1999 Mar. 1; 93(5):1658-67.
2. Jörg Schrader, Timothy T. Gordon-Walker et al., “Matrix stiffness modulates proliferation, chemotherapeutic response, and dormancy in hepatocellular carcinoma cells,” Hepatology 2011 April; 53(4):1192-205. doi:10.1002/hep.24108.
3. Britta Weigelt, Alvin T. Lo et al. Breast Cancer Research Treat (2010)
4. Mark A. LaBarge, Celeste M. Nelson et al., “Human mammary progenitor cell fate decisions are products of interactions with combinatorial microenvironments,” Integrative Biology, 2009 January; 1(1):70-9. Epub 2008 Nov. 12.
5. Justin R. Tse, Adam J. Engler et al., “Preparation of hydrogel substrates with tunable mechanical properties,” Curr Protoc Cell Biol. 2010 June; Chapter 10:Unit 10.16.
6. Justin R. Tse, Adam J. Engler, “Stiffness gradients mimicking in vivo tissue variation regulate mesenchymal stem cell fate,” PLoS One. 2011 Jan. 5; 6(1):e15978.

The above examples are provided to illustrate the invention but not to limit its scope. Other variants of the invention will be readily apparent to one of ordinary skill in the art and are encompassed by the appended claims. All references, publications, databases, and patents listed herein are hereby incorporated by reference for all purposes.

What is claimed is:

1. A combinatorial elastic modulus-modified microenvironment microarray (eMEArray) platform comprising a polymer on a substrate having a combinatorial array of cellular microenvironment components printed on said polymer and substrate, wherein the elastic modulus of the polymer mimics a specific cellular microenvironment or tissue, and wherein the cellular microenvironments elements comprising extracellular matrix, proteins, and combinations thereof.

2. The eMEArray platform of claim 1 wherein the polymer is polydimethylsiloxanes (PDMS), polyacrylamides (PA), polyurethanes, polyethylene glycol, poly(N-isopropylacrylamide), gelatin, or agarose.

3. The eMEArray platform of claim 2 wherein the polymer is polydimethylsiloxane (PDMS) or polyacrylamide (PA).

4. The eMEArray platform of claim 3, wherein the polymer is PDMS and the PDMS mimics stiffer tissues in the range of 1-10 MPa.

5. The eMEArray platform of claim 3, wherein the polymer is PA and the PA mimics softer tissues in the range of 100 Pa-100 kPa.

6. The eMEArray platform of claim 1, wherein the cellular microenvironment components selected from recombinant growth factors, cytokines, purified extracellular matrix proteins, cellular proteins and combinations thereof.

7. The eMEArray platform of claim 6 wherein the proteins are Notch 1 and 3 extracellular domains, E- and P-cadherins, Jagged1, Delta-like ligand 4, Delta serrate-like peptide, sonic hedgehog, TGFβ, EGF, PDGF, FGF, IGF, IL-6, as well as integrin-blocking and -activating antibodies, collagens type I, II, III, IV, and V, laminins I and V, fibronectin, entactin, collagenase-treated collagen 1 and 4, an combinations thereof.

8. The eMEArray platform of claim 6 wherein the cellular microenvironment components further comprising MATRIGEL.

9. The eMEArray platform of claim 1 wherein the substrate is a glass or polymer surface.

10. A method of making a combinatorial elastic modulus-modified microenvironment microarray (eMEArray) comprising the steps of: (a) preparing a printing substrata with a polymer by overlaying the polymer on the substrate surface, wherein the elastic modulus of the polymer mimics a specific cellular microenvironment or tissue; (b) preparing a master plate comprising an array of combinatorial microenvironment components; (c) printing a copy of the master plate array components onto the polymer; (d) allowing cells to bind to said array components on said polymer and washing away any unbound cells, thereby providing a combinatorial elastic modulus-modified microenvironment microarray.

11. The method of claim 10 wherein the polymer is polydimethylsiloxanes (PDMS), polyacrylamides (PA), polyurethanes, polyethylene glycol, poly(N-isopropylacrylamide), gelatin, or agarose.

12. The method of claim 11 wherein the polymer is polydimethylsiloxane (PDMS) or polyacrylamide (PA).

13. The method of claim 12, wherein the polymer is PDMS and the PDMS mimics stiffer tissues in the range of 1-10 MPa.

14. The method of claim 10, wherein the polymer is PA and the elastic modulus of the PA mimics softer tissues in the range of 100 Pa-100 kPa.

15. The method of claim 10, wherein the cellular microenvironment components selected from recombinant growth factors, cytokines, purified extracellular matrix proteins, cellular proteins and combinations thereof.

16. The method of claim 15, wherein the proteins are Notch 1 and 3 extracellular domains, E- and P-cadherins, Jagged1, Delta-like ligand 4, Delta serrate-like peptide, sonic hedgehog, TGFβ, EGF, PDGF, FGF, IGF, IL-6, as well as integrin-blocking and -activating antibodies, collagens type I, II, III, IV, and V, laminins I and V, fibronectin, entactin, collagenase-treated collagen 1 and 4, an combinations thereof.

17. The method of claim 15, wherein the cellular microenvironment components further comprising MATRIGEL.

18. The method of claim 10 wherein the substrate is a glass or polymer surface.

19. The method of claim 10 wherein the cells are any epithelial, stem, or progenitor cells.

20. A method of screening cellular response to a drug comprising the steps of: (a) providing a combinatorial elastic modulus-modified microenvironment microarray (eMEArray) as prepared in claim 10; (b) incubating said eMEArray; (c) contacting a drug with the cells and the eMEArray; (c) detecting any change in the cell.
